(19) United States
(12) Patent Application Publication
(10) Pub. No.: US 2012/0129224 A1
(43) Pub. Date: May 24, 2012

(54) Method and apparatus for changing one type of cell into another type of cell

(76) Inventor: Chauncey B. Sayre, Huntington Beach, CA (US)

(21) Appl. No.: 13/288,801

(22) Filed: Nov. 3, 2011

Related U.S. Application Data
(63) Continuation of application No. PCT/US2010/033728, filed on May 5, 2010.
(60) Provisional application No. 61/176,643, filed on May 8, 2009.

(51) Int. Cl. C12P 19/34; C12M 1/42; C07K 1/14; C12N 15/85; C12N 13/00

(52) U.S. Cl. 435/91.21; 435/455; 435/173.1; 530/412; 435/283.1

(57) ABSTRACT

A method and apparatus converts host cells of a first type into cells of a second type when the host cells are placed in intimate contact with donor cells of the second type. Under predetermined conditions there is transport of a sufficient number of mRNA molecules from the donor cells into the host cells to reprogram the host cells into the second type. The host and donor cells may be subjected to while in intimate contact to a transporting force that enables the mRNA molecules of the donor cells to penetrate an outer membrane wall of host cells without damaging the membrane wall. The transporting force may include an electric field, a magnetic field, or a combined electric field and magnetic field.

### RELATED PATENT APPLICATIONS & INCORPORATION BY REFERENCE

This application is a continuation patent application based on international patent application number PCT/US2010/33728, filed May 5, 2010, which claimed the benefit under 35 USC 119(e) of U.S. Provisional Patent Application No. 61/176,643, entitled “METHOD OF AND APPARATUS FOR CHANGING ONE TYPE OF CELL INTO ANOTHER TYPE OF CELL,” filed May 8, 2009. This related provisional patent application is incorporated herein by reference and made a part of this application. If any conflict arises between the disclosure of the invention in this PCT application and that in the related patent application, the disclosure in this PCT application shall govern. Moreover, any and all U.S. patents, U.S. patent applications, and other documents, hard copy or electronic, cited or referred to in this application are incorporated herein by reference and made a part of this application.

### DEFINITIONS

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

The words “substantially” and “essentially” are intended to be equivalent in meaning

The word “cytoplasm” means the material of a cell between the nucleus of the cell and the outer membrane wall of the cell.

The words “outer membrane wall” means the biological membrane separating the interior of a cell from the outside environment.

The word “ova” are mature female reproductive cells that can divide to give rise to an embryo.

The word “polyadenylation” means the addition of a poly(A) tail, a stretch of RNA where all the bases are adenines, onto an RNA molecule.

The words “viral vector” mean a type of virus used in cancer therapy where a virus is changed in the laboratory and cannot cause disease.

The words “differentiated cells” means cells derived by a process by which a less specialized cell becomes a more specialized cell type.

The words “pluripotent cell” means a cell that is able to differentiate into many cell types.

### BACKGROUND

Different types of cells have different functions, for example, a beta cell of the pancreas secrets insulin and a cardiac cell of the atrium has a fluid action potential and contracts rhythmically. Converting one type of cell into another type of cell without any deleterious effects is a long sought after goal of molecular biologists. Also, such converted cells may be used in the medical treatment of numerous human and animal afflictions. But current methods for making such cells introduce into their structure substances that may be potentially harmful to a patient. For example, in converting differentiated cells into pluripotent cells, such techniques as chemicalporation, lypofection and plasmid utilization have been employed that introduce deoxyribonucleic acid (DNA) into host cells. This DNA has objectionable residual effects. Some have used viruses or viral vectors that again have objectionable residual effects. Examples of deleterious substances are foreign aliphatic compositions, metals, or chemicals that porate or otherwise damage the outer membrane wall of the cells. The processes used may also have negative effects.

Ribonucleic acid (RNA) plays an important role in the growth of cells. RNA molecules are smaller than DNA molecules and usually are single-stranded, while DNA molecules are usually double-stranded. There are a wide variety of RNA molecules including messenger ribonucleic (mRNA) and micro ribonucleic (miRNA) molecules. The mRNA molecule encodes a chemical “blueprint” for a protein product. mRNA is transcribed from a DNA template, and carries coding information to the sites within a cell of protein synthesis: the ribosomes. Here, a nucleic acid polymer is translated into a polymer of amino acids: a protein. The resulting proteins determine the type of living cell being produced. The miRNA regulate gene expression. miRNA molecules are partially complementary to one or more mRNA molecules, and their main function is to down-regulate gene expression. The mRNA and miRNA molecules include phosphate groups along their backbones that give these segments a negative charge.

### SUMMARY

My method and apparatus of converting host cells into different cell types have one or more of the features depicted in the embodiments discussed in the section entitled “DETAILED DESCRIPTION OF SOME ILLUSTRATIVE EMBODIMENTS.” The claims that follow define my method and apparatus, distinguishing them from the prior art; however, without limiting the scope of my method and apparatus as expressed by these claims, in general terms, some, but not necessarily all, of their features are:

One, in my method of converting host cells of a first type into cells of a second type the host cells are placed in intimate contact with donor cells of the second type. The intimate contact is under predetermined conditions that transport a sufficient number of mRNA molecules from the donor cells into the host cells to reprogram the host cells into the second type. The conditions may include the application of an electromagnetic force, using a media that enables the electromagnetic force to produce an electrophoresis effect that acts on the mRNA molecules from the donor cells to transport them through an outer cell membrane into the hosts cells, subjecting the host and donor cells while in intimate contact to a transporting force that enables the mRNA molecules of the donor cells to penetrate an outer membrane wall of host cells without damaging the membrane wall. The transporting force may include an electric field, a magnetic field, or a combined electric field and magnetic field.

Two, my method of converting host cells of a first type into cells of a second type may include placing the host cells in intimate and direct contact with an activated ova in a matrix and subjecting the matrix to electrophoresis to transport an array of reprogramming substances being produced by the activated ova across an outer membrane of individual host cells into the host cell to reprogram the host cells.

In one embodiment, my method of transferring a biologically active material to host cells comprises the steps of

(a) placing the biologically active material on a first electrode of a pair of electrodes and placing on a second electrode of the pair of electrodes the host cells, and

(b) positioning the electrodes closely together so there is a narrow gap between the electrodes, and

(h) applying an electrical field across the closely spaced electrodes that has sufficient strength so that the biologically active material migrates across the gap and into the host cells on the second electrode.

The gap does not exceed 50 microns, and may be, for example, substantially from 15 microns to 5 millimeters. The strength of the electric field may be, for example, substantially from 50 to 150 volts. A static direct electric field or a pulsating direct electric field may be applied across the electrodes. For example, the magnetic field may be applied across the gap concurrent with the application of the electric field, the direction of the magnetic field being substantially at a right angle to the direction of migration across the gap of the biologically active material. A gel may be disposed between the electrodes. The biologically active material may applied as a thin coating carried by the first electrode that has a thickness substantially from 0.015 to 4.0 millimeters and the host cells are applied as a thin coating carried by the second electrode that has a thickness substantially from 0.015 to 4.0 millimeters, the coatings being substantially planar and facing each other and the first electrode having a negative polarity and the second electrode having a positive polarity. The biologically active material may comprise activated ova that produces a mixture mRNA and miRNA in predetermined proportions. The biologically active material comprises a mixture of mRNA and miRNA molecules that are amplified in number from those originating from the activated ova, the amplified in number of mRNA and miRNA molecules being substantially in the same proportions as normally yielded by the activated ova.

Three, my method also includes changing differentiate host cells into pluripotent cells, and may comprise the steps of

(a) extracting mRNA and miRNA from an activated ova of a living organism when the ova is reprogramming its nucleus,

(b) amplifying the number of molecules of mRNA and miRNA extracted from step (a),

(c) processing the mRNA and miRNA from step (b) by polyadenylation,

(d) placing the polyadenylated mixture of mRNA and miRNA on a negative electrode of a pair of electrodes and placing on a positive electrode of the pair of electrodes differentiate host cells to be transformed into the pluripotent cells, and

(e) positioning the electrodes closely together so there is a narrow gap between the electrodes, and

(f) applying an electrical field across the closely spaced electrodes that has sufficient strength so that the mRNA and miRNA migrate across the gap into the host cells on the second electrode to interact therewith to transform the host cells into the pluripotent cells.

In my method changing differentiate host cells into pluripotent cells, subsequent to step (c) and prior to step (d), the mRNA and miRNA molecules are blended in predetermined proportions substantially in the same proportions as normally yielded by the activated ova. The ova may be chemically, electrically or mechanically stimulated to make the mRNA and miRNA. The mRNA and miRNA may be extracted using a centrifuge. The mRNA and miRNA may be charged negatively so they migrate to the positive electrode. In step (b) the mRNA and miRNA may be hydrolyzed in an aqueous solution, purified and freeze-dried prior to step (c). My method changing differentiate host cells into pluripotent cells may use a gel material within the gap, the gel material being selected from the group consisting of agarose, Matrigel, and acrilamide. The gel material may include an electrolyte, and my method may be conducted without the use of harmful substances that would impede clinical use.

Four, my method includes separating the constituents of a mix of reprogramming substances including reprogramming proteins, mRNA and miRNA. It comprises the steps of

(a) providing a gel within a light-transmitting container that has a closed end and an open end.

(b) including makers within the container that identify by color separate zones along the length of the container corresponding to collection of separated constituents, and

(c) under cryogenic temperature conditions, placing the mix in contact with the gel near the open end and centrifuging the container so the constituents separate and collect in different layers within the gel according to their mobility.

My apparatus for transferring biologically active material to host cells is suitable for conducting my method. It comprises a support mounting member, a pair electrodes attached to the mounting member, and a power source. The electrodes have opposed planar surfaces facing each other. At least one electrode is moveably mounted so the electrodes have (a) a first, spaced apart position to enable the biologically active material to be placed on the planar surface of one electrode and the host cells to be placed on the planar surface of the other electrode, and (b) a second position where the electrodes are close together so there is a gap between the electrodes that does not exceed 50 microns. The power source applies an electrical field across the electrodes when in the second position that has sufficient strength so that the biologically active material migrates across the gap into the host cells on the second electrode. The electrodes may be substantially parallel plates. The apparatus may include means for applying a magnetic field across the gap concurrent with the application of the electric field. The direction of the magnetic field may be substantially at a right angle to the direction of migration across the gap of the biologically active material.

These features are not listed in any rank order nor is this list intended to be exhaustive.

### DESCRIPTION OF THE DRAWING

Some embodiments of my method and apparatus are discussed in detail in connection with the accompanying drawing, which is for illustrative purposes only. This drawing includes the following figures (Figs.), with like numerals indicating like parts:

FIG. 1 is a schematic illustration of my apparatus for transferring biologically active material to host cells.

FIG. 1A is a schematic illustration showing the electrodes of my apparatus depicted in FIG. 1 spaced close to each other with one electrode carrying biologically active material and the other electrode carrying host cells.

FIG. 2 is a schematic illustration showing the electrodes of my apparatus depicted in FIG. 1 spaced far apart with a thin layer of biologically active material applied to a surface of the negative electrode and a thin layer of host cells applied to a surface of the positive electrode that faces the negative electrode.

FIG. 2A is a cross-sectional view taken along line 2A-2A of FIG. 2.

FIG. 2B is a cross-sectional view taken along line 2B-2B of FIG. 2.

FIG. 3 is a schematic illustration showing the electrodes of my apparatus depicted in FIG. 1 spaced close to each other with a coating of biologically active material in contact with a coating of host cells.

FIG. 4 is an enlarged schematic illustration showing a biologically active material of mRNA and miRNA being transported into the host cells.

FIG. 5A is a schematic side view of an apparatus for segregating constituents of a mix of different biologically active materials prior to centrifuging.

FIG. 5B is a plan view taken along line 5A-5A of FIG. 5.

FIG. 5C is a schematic side view of an apparatus shown in FIG. 5A after centrifuging.

### DETAILED DESCRIPTION OF SOME ILLUSTRATIVE EMBODIMENTS

### General

My method enables biologically active material to be transferred into the nucleus of host cells in a manner to change the host cells into a new type of cell and avoid introducing harmful materials into the host cells or damaging the host cells. This transfer is achieved under the influence of an electromagnetic force, for example, an electrical field established between a pair of electrodes. The biologically active material, however, is essentially devoid of genetic substances such as DNA. A characteristic of the molecules of the biologically active material is their mobility in the electrical field as compared to DNA, or other unwanted and potentially harmful larger molecules that may be present. In one embodiment (direct transfer method) the biologically active material is the material being produced by donor cells and directly transferred to the host cells from the donor cells while in intimate contact. In another embodiment (indirect transfer method) the biologically active material is a mixture of the more mobile, smaller molecules derived from donor cells but the donor cells are not in contact with the host cells. The biologically active material may include (1) those proteins that reprogram the nucleus of host cells or (2) mRNA or (3) a mixture of mRNA and miRNA or (4) a mixture of mRNA, miRNA and the reprogramming proteins.

In the direct transfer method the material being transported into the host cells comprises the mobile, smaller molecules: the mRNA and miRNA and some reprogramming proteins. For example, the biologically active material in the direct transfer method may be the material derived during the early stages after activation of an ova from a living organism. Activation may be achieved without fertilization or the ova may be fertilized. The ova is activated in conventional ways, for example, chemically, electrically or mechanically. The ova donor cells may be animal and the host cells human. The host cells are in intimate contact with the activated ova cells in a matrix that facilitates electrophoresis. While in such intimate contact, the matrix is subjected to electromagnetic conditions to induce electrophoresis within the matrix to transport an array of reprogramming substances being produced by the activated ova across the outer membrane wall of individual host cells. These reprogramming substances have the mobility that any undesirable material from the ova cells lack. Consequently, they separate from any undesirable material and pass through the outer membrane wall of the host cell to reprogram the host cells. Normally, the ova are unfertilized and may be from the same species as the host cells, but they do not necessary have to be, for example, bovine and porcine ova may be used to provide mRNA and miRNA and reprogramming proteins that are introduced into human host cells.

In the indirect transfer method, the reprogramming substances (reprogramming proteins, mRNA and miRNA) are (1) an extracted mix separated from donor cells and transported into the host cells under the influence of an electromagnetic force or (2) reprogramming proteins, mRNA and miRNA from donor cells that are isolated and segregated and either individually, or in various combinations, are transported into the host cells under the influence of an electromagnetic force. The host cells of a first type are thus converted into cells of a second type depending on the chemical nature of these reprogramming substances. By placing the reprogramming substances in intimate contact with the host cells under suitable conditions, a sufficient number of the reprogramming substances migrate into the host cells to covert them into cells of a different type, such as differentiated cells into pluripotent cells. Such conditions include subjecting the reprogramming substances and host cells to electromagnetic energy while in intimate contact. In the indirect transfer method the biologically active material may be (1) substantially only the array of reprogramming proteins from the donor cells, (2) substantially only the mRNA from the donor cells, (3) substantially only a mixture of such donor cell mRNA and miRNA, or (4) a mix of all these from the donor cells. In any such mixture or mix the proportion of 7 constituents may be controlled so that the amount of each constituent is close to that which is present in the donor cells. In accordance with one feature of the indirect transfer method, the constituents of a starting mixture or mix derived from any source may be separated into individual constituents as subsequently discussed in greater detail.

In both embodiments, after transfer of the biologically active material into the hosts cells, these cells are cultured for a sufficient time, for example several hours, for the host cells to change type. In both embodiments, the change in the host cells occurs without any significant adverse impact on the now converted host cells and without the use of detrimental materials or production procedures. Either embodiment of my method may be used to change differentiate host cells into pluripotent cells. For example, both embodiments of my method yields pluripotent cells characterized as having the potential to differentiate into a more diverse range of cells than pluripotent cells produced by known techniques. Potentially any viable cell type in a human or animal body may be a host cell or a donor cell, for example, spermatogonial, hESC, mesenchymal, hematopoetic, or liver cells. The reprogrammed cells produced by my method may be used clinically by medical practitioners depending on the nature of the disease being treated, and both embodiments may be useful for clinical purposes. (Here donor and host cells are usually of the same species).

In both embodiments of my method, a media may be used that enables the electromagnetic energy to produce an electrophoresis effect that acts on the molecules of the biologically active material to transport them through an outer membrane wall of a host cell into the cytoplasm surrounding the nucleus of the host cell. This, for example, enables the mobile molecules to pass through the outer membrane wall of host cells without damaging these membrane walls. The transporting force may be an electric field, or a combined electric field and magnetic field. The magnetic field accelerates transfer. Freeze drying may be advantageously employed in both embodiments of my method. RNA for the host cells or the proteins for the host cells may be extracted from the cell type into which one wishes to convert the host cells. The donor cells are placed in a lyophilizer to dehydrate the donor cells and kept them at a very cool temperature to allow for freeze drying that maintains the shape and integrity of the molecules. When most of the water has been removed through freeze-drying, this material may now be coated onto a dielectric layer as subsequently discussed.

The unique pluripotent cells produced by my method may subsequently be converted into differentiated cells by introducing into these cells mRNA or a mixture of mRNA and miRNA of selected differentiated cells. These newly constituted differentiated cells derived from pluripotent cells produced by my method are then injected into a patient. The selected differentiated cells providing the RNA do not have to be derived from the same species as the pluripotent cells; however, in some circumstances it may be best that they are of the same species, and mostly that they be both mammalian. Typical differentiated donor cells from which the RNA is extracted may be from potentially any viable cell type in a human body, including brain cells, retinal cells, spleen cells, heart cells, nerve cells, pancreatic cells, placental cells, ovarian cells, and epidermal cells. Consequently, donor's differentiate cells of one predetermined type may be used to make the pluripotent cells according to my method, and then these pluripotent cells may be the starting material to make differentiated cells of another type using RNA from selected differentiated cells from the same or a different donor.

In one embodiment of my apparatus, the electric field is provided by a pair of electrodes, at least one electrode being movable with respect to the other electrode. The source of the biologically active material is placed on one electrode and the host cells are placed on the other electrode. An electrical insulating matrix is between the electrodes so, when they are in close proximity, they form an electrical capacitor structure that enables the biologically active material to migrate into the hosts cells upon application of a voltage across the electrodes. For example, the biologically active material may be applied as a thin coating to an exposed surface of a layer of dielectric insulting material that is mounted on a negative electrode and the host cells may be applied as a thin coating to an exposed surface of a layer of dielectric insulting material that is mounted on a positive electrode. The electrodes are pressed against each other with coatings of the biologically active material and the host cells in intimate contact. Then the voltage is applied across the electrodes at a sufficient level to cause migration of the biologically active material into the host cells without essentially any current flow between the electrodes. When the electrodes are in close proximity, there is only a narrow gap between them that is minimal but sufficient to suppress any current flow across the electrodes when voltage is applied. The electrical insulating matrix may be a gel-type substance that serves as a matrix and conduit for molecules of interest—specifically RNA. Suitable gel-type substances are, for example, agarose, Matrigel (a trademark of BD Biosciences, and acrilamide. An electrolyte, for example, a phosphate buffered saline (PBS) solution may be included in the biologically active material or in the matrix.

The electrodes may be planar having surfaces that face each other. One or both electrodes may initially be mounted to move and are separated by a distance greater than, for example, 6 inches to allow formation of the dielectric layers and to apply to their respective electrodes the coatings of the biologically active material and host cells. Then, the electrodes are moved towards each other with the layers touching but the electrodes separated by a minimal distance to form a gap between the electrodes. An electrical field is now applied across the closely spaced electrodes that typically is substantially from 50 to 150 volts. The electric field is direct, and may be a continuous, static direct electric field or a pulsating direct electric field that intermittently applies the electric field across the electrodes.

The gap between the electrodes typically does not exceed approximately 5 millimeters, and may be substantially from 15 microns to 5 millimeters. Optionally, a magnetic field may be applied across the gap concurrent with the application of the electric field. The direction of the magnetic field is substantially at a right angle to the direction of the electric field, which is substantially the direction of migration across the gap of the biologically active material. The thickness of each dielectric layer typically does not exceed 2 millimeters and typically is substantially from 0.05 to 4 millimeters. The thickness of each coating typically is substantially from 0.015 to 4.0 millimeters. The spacing between the electrodes and voltage applied across these electrodes is controlled so that essentially no current flows between the electrodes across the narrow gap. Rather, because of the negative polarity of the electrode carrying, for example, the mRNA and miRNA and the negative charged phosphate groups along the backbones of the mRNA and miRNA, the mRNA and miRNA molecules are repelled by this electrode when power is supplied, propelling the mRNA and miRNA molecules across the gap through the outer membrane wall into the interior of the host cells.

As discussed above, the source of the mRNA and miRNA may be direct from donor cells, or may be derived from other sources. Reprogramming substances derived from sources other than directly from donor cells may comprise a mix of constituents including reprogramming proteins, mRNA and miRNA. This derived mix of mRNA and miRNA may be in substantially the same proportions produced by an ova in vivo within the body of the donor of the ova. The quantity of mRNA and miRNA in the derived mixture may be amplified in accordance with conventional procedures set forth in Section A. Moreover, in my indirect transfer method, though produced in vitro, the mRNA and miRNA reprogram the genome of the host cell in essentially the same way donor cells would in my direct transfer method. The mRNA and miRNA decompose much faster than DNA, and therefore have no objectionable residual effects. Consequently, if anything, for an economically viable manufacturing process, the effective life of the mRNA and miRNA should be extended. The mRNA and miRNA molecules are also subject to enzyme attack, and this should be minimized. In order to slow degradation of the amplified mRNA and miRNA molecules, these molecules may be processed by polyadenylation. This polyadenylated mixture may be placed on the negative electrode and the host cells to be transformed into the pluripotent cells may be placed on the positive electrode, and then the procedures discussed above are followed.

In the Example 1 below, the mRNA and miRNA, and reprogramming proteins, are directly transferred from the ova to the differentiate host cells to be converted into pluripotent cells in accordance with my direct transfer method. For larger scale production of the pluripotent cells, my indirect method may be used were the number of molecules of the mRNA and miRNA used is greatly amplified in accordance with conventional methods. This provides a biologically active material that is a mixture of concentrated mRNA and miRNA in an aqueous solution that is purified and separated from other types of RNA and other unwanted chemicals. In the mixture of amplified mRNA and miRNA, the mRNA and miRNA molecules are in substantially the same proportions as produced by their complementary activated ova. The mRNA is able to be amplified 5 million-fold and the miRNA is able to be amplified 1,000-fold. The ratio of mRNA to miRNA varies depending on the application. Typically, after amplification the mRNA and miRNA are mixed according to their natural proportions occurring in the cell.

Reprogramming substances provided without direct contact from donor cells may comprise a mix of constituents including organelles, reprogramming proteins, mRNA and miRNA. These constituents are separated and then the separated constituents are used individually, or combined in any suitable re-mixture or new combination, and used in the indirect transfer method discussed above. This separation method includes providing a separating gel within a light-transmitting container that has a closed end and an open end. The separating gel includes circumferential, color coded markers that are viewed through the light-transmitting container that identify by separate color zones along the length of the container the location where corresponding separated constituents collect within the separating gel under the influence of centrifugal forces. The separating gel forms under cryogenic temperature conditions, the mix of constituents is placed in contact therewith near the open end of the container and then spun, centrifuging the container so the constituents and kinetically separate and collect in different layers within the separating gel according to their mobility.

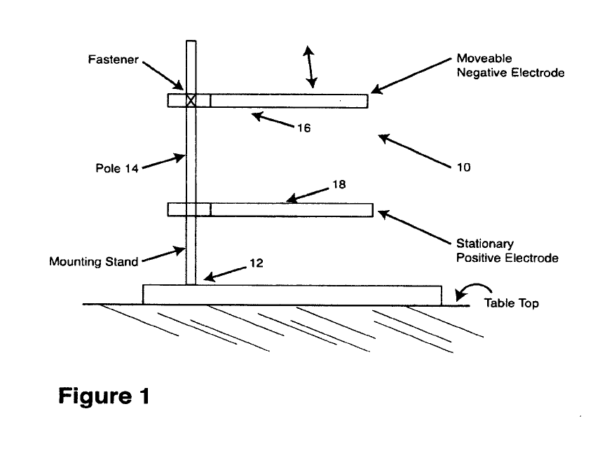

### FIGS. 1 Through 4

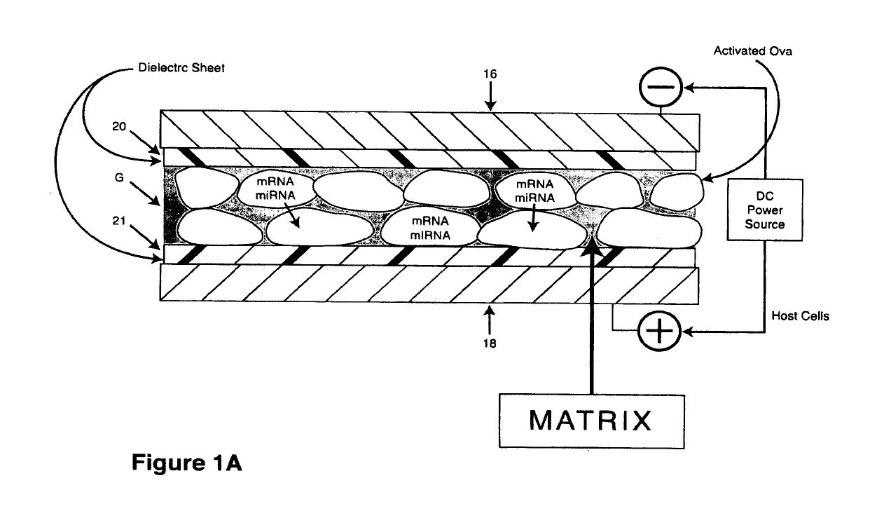

Referring to FIGS. 1 through 4, my apparatus 10 includes a stand 12 having a vertical pole 14 to which are attached a pair of disk shaped electrodes 16 and 18 connected across the positive and negative terminals of a DC power source (FIG. 1A). Each electrode 16 and 18 carries on an inside surface 16a (FIG. 2A) and 18a (FIG. 2B), respectively, a thin dialectic layer 20 and 21, respectively. The electrodes 16 and 18 typically are made of gold and have a thickness substantially from 0.25 to 1.0 millimeters and a diameter substantially from 15 to 20 millimeters. When apart, a thin coating C1 of the biologically active material being transferred is applied to the exposed surface of the dialectic layer 20 and a thin coating C2 of the host cells is applied to the exposed surface of the dialectic layer 21. These coating C1 and C2 are substantially from 0.015 to 4.0 millimeters thick. Initially, the electrodes 16 and 18 are parallel to each other and held generally in a horizontal orientation wide apart, for example, 6 inches, with the coated dialectic layers 20 and 21 facing each other. After applying the biologically active material and host cells, the electrodes 16 and 18 are positioned to be very close to each other but do not touch. As illustrated, the electrode 16 is the movable electrode that is repositioned after applying the biologically active material to this electrode. The host cells may be applied to the stationary electrode 18 when the electrodes 16 and 18 are in the separated position.

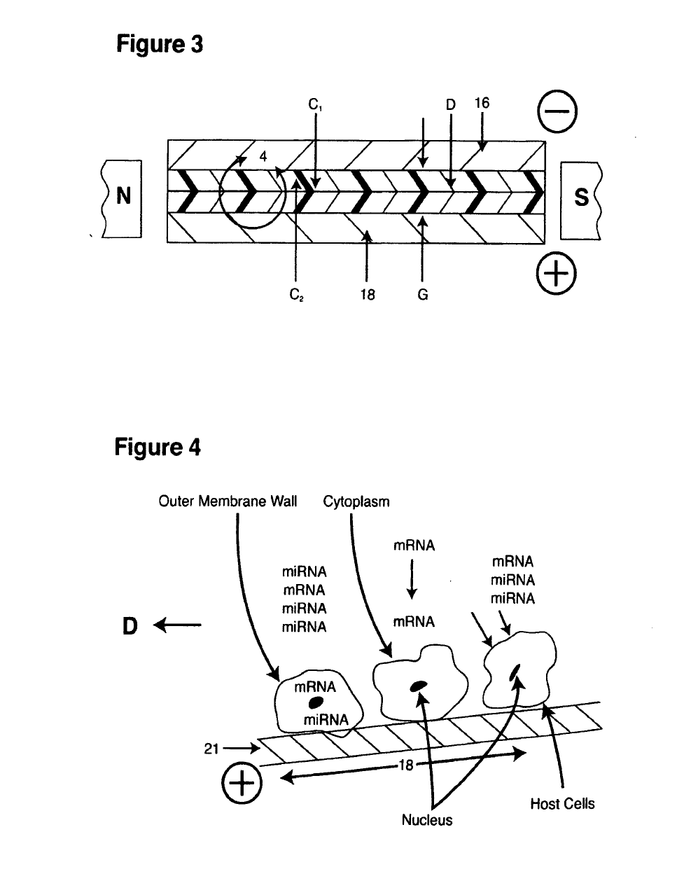

As shown in FIG. 1A, with the electrode 16 being negative and the electrode 18 being positive and these electrodes in close proximity so only a narrow gap G (FIG. 1A) of about 50 microns or less is between the electrodes and the coatings C1 and C2 on the dialectic layers 20 and 21 touching and in intimate contact, a direct voltage, which may be pulsed, is applied across the electrodes. In the Example I discussed below, the voltage was substantially from 50 to 150 volts and essentially no current flowed between the electrodes 16 and 18. In effect, the assembly of electrodes 16 and 18 and the layers 20 and 21 with the coating C1 and C2 thereon function as a biological vectoring device. As illustrated in FIG. 1A, the material between the electrodes forms a matrix M in which, at least in part due to electrophoresis, the biologically active material, as illustrated in FIG. 4, is transported through an outer membrane wall of a host cell into the cytoplasm surrounding the nucleus of the host cell. The assembled electrodes 16 and 18 with, for example, mRNA and miRNA molecules from an activated ova move in the electric field in the direction of the arrow A that is substantially perpendicular to the planar surface 18a and through the outer membrane wall into the interior of the host cells to render these hosts cells pluripotent.

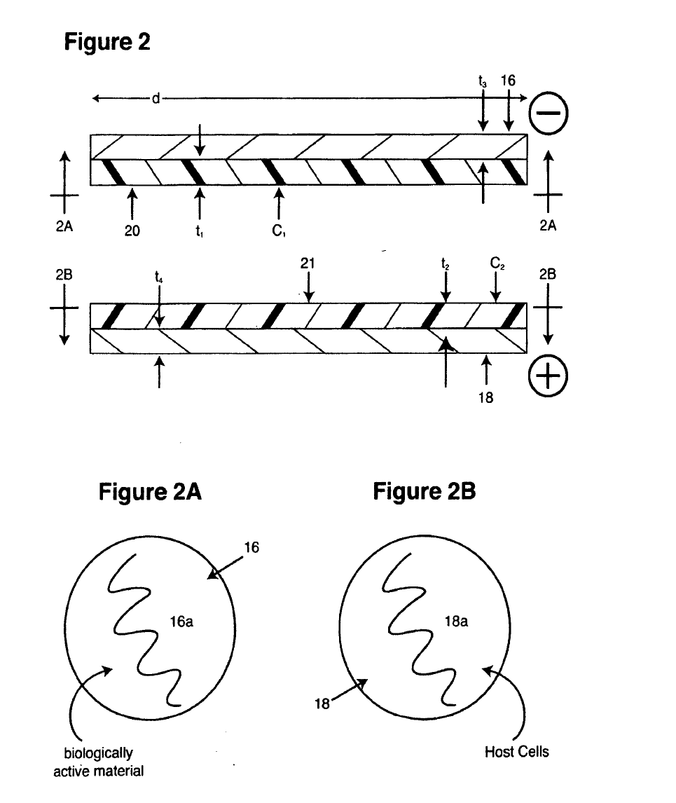

FIG. 2 shows the electrodes 16 and 18 widely separated with the host cells applied as a thin layer to the surface 18a and, for example, an amplified and polyadenylated mixture of mRNA and miRNA molecules in substantially the same proportions as produced by their originating activated ova applied as a thin layer to the surface 16a. The electrode 16 is moved to engage the electrode 18 as depicted in FIG. 3, with the surface of the mixture layer in intimate contact with the surface of the host cells layer. Concurrent with the application of an electric field, a magnetic field is applied by a magnet 26 by positioning the assembled electrodes 16 and 18 between the North and South poles of the magnet. The direction of the magnetic field is indicated by the arrow D in FIG. 3 pointing from the South pole to the North pole of the magnet 26. The direction D of the magnet field along the gap G is substantially at a right angle to the direction A of the electric field and in general of the direction of movement of the mRNA and miRNA molecules in the electric field. Electrical energy from the DC power source is applied in pulses over time period of, for example, from 60 to 90 seconds duration. During this time period a sufficient number of molecules the biologically active material enter the host cells to initiate their transformation into a different type of cell than the host cell depending in the character of the biologically active material being transported into the host cells. Then the electrodes 16 and 18 are separated and these treated host cells collected and incubated to allow the transformation into the new cells over an extended time period, for example, from 24 to 72 hours.

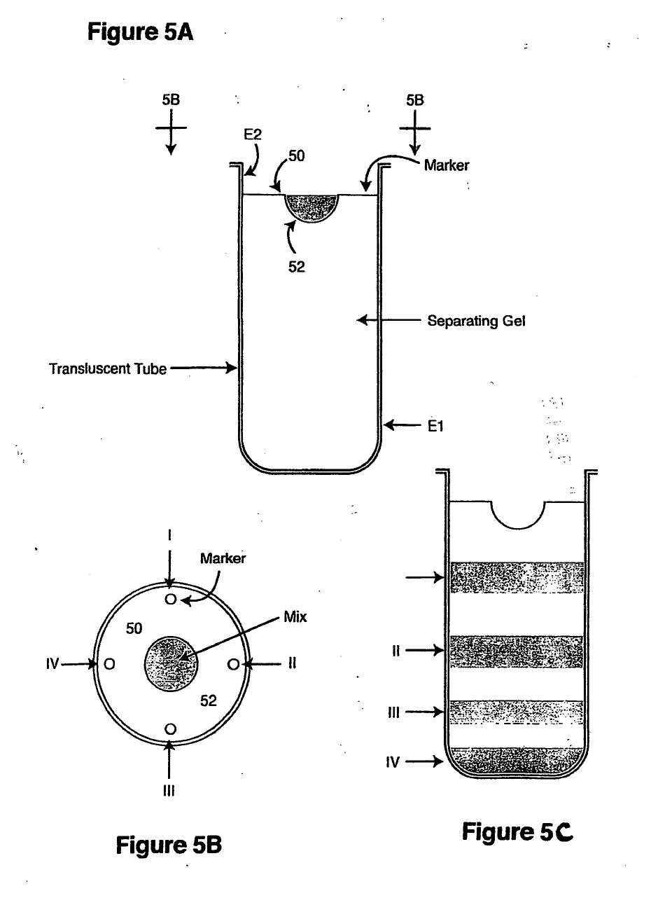

### FIGS. 5A Through 5C

FIGS. 5A through 5C depicted separating into individual constituents the reprogramming substances derived from sources other than directly from donor cells as discussed in Example 1 through 3. These constituents—reprogramming proteins, mRNA and miRNA—after separation are used individually, or combined in any suitable re-mixture or new combination, and used in the indirect transfer method discussed above. In this separation method a separating gel is held within a light-transmitting container, for example the translucent tube that has a closed end E1 and an open end E2. Embedded in the exposed top surface 50 of the mass of separating gel about the circumference are color-coded markers I, II, III and IV. These markers I, II, III and IV will move into and identify distinct layers or color zones upon centrifuging the tube according to their mobility with the separating gel; for example, one marker may correspond to the reprogramming proteins, another to mRNA, another to miRNA and yet another to DNA. Prior to centrifuging the tube, the mix of constituents is placed a shallow well 52 formed in the top surface 50 near the open end of the container and then the tube is spun so the constituents kinetically separate and collect in different layers within the separating gel according to their mobility. Since the markers correspond to the different constituents, these different constituents are distributed accordingly. One may view these markers in the separate zones after centrifuging through the light-transmitting tube. Thus the separated constituents are within and identified by the separate color zones along the length of the container.

The separating gel forms under cryogenic temperature conditions and may comprise use two fluorocarbons: perflurodecaline and perfluoro-proplamine that are used as a base for artificial blood. These fluorocarbons are very inert and do not interact with biological material. They are mixed in a 50:50 ratio, vigorously mixed and then placed in the tube, which is placed in dry ice or liquid nitrogen and kept at −79.2° C. to form the separating gel. The best accurate segmentation of the constituents of the mix occurs after centrifugation at −70° C. When the constituents are separated into the layers, the separating gel while in the semi-solid state is removed as a unity piece that is then sliced into sections to segregate the constituents.

The following are examples of practicing my method.

### Example I

### Materials and Equipment:

Activated ova-1,000 porcine and 1,000 bovine oocytes (Applied Reproductive Technologies, Madison Wis.)
Hepatocytes (human liver cells) (CellzDirect, Durham N.C.)
CHEK kit (CellzDirect)
Matrigel (BD Biosciences, San Jose Calif.)
The electrophoresis apparatus depicted in FIGS. 1 through 4.

Two electrodes at room temperature were taken and coated with Matrigel. The Matrigel was removed from −25 degree Celsius storage and placed on ice under a biosafety cabinet. The Matrigel was placed onto the facing surfaces of both electrodes at a concentration of 50 microliters (ul) per cm², and both electrodes were incubated at 37 degrees Celsius for 30 minutes. A vial of approximately 6 million cryopreserved human hepatocytes was thawed according to the CellzDirect protocol as explained in Section B and applied as a coating to the Matrigel layer on the positive electrode. The porcine and bovine oocytes were transferred to the top of the Matrigel-coated electrode that had been covered with hepatocytes. Then the negative electrode was gently placed on top of the oocytes and pressure was applied. Next different electrical pulses were applied: one at 25 volts, one at 100 volts, one at 125 volts. These pulses were pulsed repeatedly and lasted for approximately one second intervals for a time period of approximately three minutes. The gold electrodes were removed and placed in a 35 mm dish containing media (Media First Day-Conception Technologies of San Diego, Calif.) that promotes oocyte growth in its early stages. The next day, the media was changed to Media Second Day (Conception Technologies). The third day, the cells were transferred to Media Day Three (Conception Technologies). On the fifth day, the cells were transferred to Media Day Five (Conception Technologies). The next day (Day 6), the excess media was siphoned off and 2 milliliters (ml) of phosphate buffered saline (PBS) were added to the dishes and vigorously mixed. This washing process was repeated three times. The cells were blocked with 6 ml of 4% normal Goat Serum in PBS and incubated at room temperature for 30 minutes. The excess goat serum was aspirated. The cells were then stained with five different sets of primary antibodies known to indicate pluripotency from Chemicon, part of Millipore in Temecula, Calif. These included: SSEA-3 (IgM), SSEA-4 (IgM), TRA-1-60 (IgM), TRA-1-81 (IgM), and Oct-4 (IgG). The antibodies were allowed to attach for two hours, and excess primary antibodies were then thoroughly washed off with 10 ml DPBS. Secondary antibodies from Millipore, including anti-SSEA-3, anti-SSEA-4, anti-TRA-1-60, anti-TRA-1-81, and anti-Oct-4 were added. The anti-SSEA-3, anti-SSEA-4, and anti-Oct-4 secondary antibodies were conjugated to FITC, which fluoresces green. The anti-TRA- and anti-TRA-1-81 were conjugated to Cy5, which fluoresces red. The antibodies were incubated with the cells for one hour at room temperature. The cells were washed with 10 mL DPBS to remove excess secondary antibodies. The cells were then separated from the 12.5 millimeters (mm) disc, spun down, and placed in a well, where a group of them fluoresced green, indicating the presence of any or all of three genes, including SSEA-3, SSEA-4 and Oct-4, which indicate pluripotency. This fluorescence was captured by camera.

### Example II

### Materials and Equipment:

Cryopreserved hepatocytes (human liver cells CellzDirect),
Rat Tail Collagen Type I (BD Biosystems),
Total Human Placental RNA Solution (100 micrograms Total Human Placental RNA in 100 microliters suspension fluid—Applied Biosystems),
CHEK kit (CellzDirect)
Gene Pulsar XL, Bio-Rad Laboratories of Hercules, Calif.

To begin, six conductive gold electrodes were placed in individual wells of a 24 well plate, and then coated with Rat Tail Collagen Type I. 1500 microliters of collagen solution diluted to 50 micrograms/milliliters with 0.02 Normal acetic acid were prepared, and 250 microliters of this solution was placed in each well on top of the gold electrodes. The electrodes were incubated at room temperature for one hour allowing the collagen solution to harden on the surface of the electrode, and any excess remaining solution was aspirated. The electrodes were rinsed with PBS with calcium and magnesium to remove the acid. This rinse was repeated three times. Then 7.9 million human hepatocytes (CellzDirect) in a vial of were then thawed according to standard procedures in a 37 degree water bath. The cells were carefully washed with CHRM medium, centrifuged at 100× gravity for 10 minutes at room temperature, and resuspended in 2.5 ml of thawing/plating medium. 500 uL of the hepatocyte solution was placed on each electrode and the electrodes were incubated in a 37 degree 5% CO2 incubator for 6 hours. After this period, the thawing/plating medium was aspirated from each well, and replaced with incubation medium warmed to 37 degrees. The electrodes were returned to the incubator and left to recover overnight. After approximately eighteen hours, the gold electrodes were carefully removed from the wells with tweezers and placed in a 4 mm cuvette of the Gene Pulsar XL. The cuvette was filled with 100 microliters of the Total Human Placental RNA Solution and then the cuvette was placed in the chamber of the Gene Pulsar XL and three electrical pulses of 160V were applied for 1500 milliseconds at 500 microFarads, and infinite resistance. The hepatocytes plated on the gold electrodes were delicately removed from the cuvette with tweezers and returned to the individual wells. The cells were incubated at 37 degrees C. for six hours. After about 72 hours these hepatocytes became more translucent and histologically began to resemble human placental cells.

### Example III

### Materials and Equipment:

Cryopreserved human embryonic stem cells/hESC
(Invitrogen), CELLstart
StemPro hESC SFM (Invitrogen),
Total Human Liver RNA (100 micrograms Total Human Liver RNA in 100 microliters suspension fluid—Applied Biosystems)
Gene Pulsar XL, Bio-Rad Laboratories of Hercules, Calif.

Six gold electrodes were coated and then placed in individual wells with CELLstart diluted 1:50 in Dulbecco's Phosphate Buffered Saline with calcium and magnesium. 160 uL of the diluted solution was placed in each well and incubated at 37 degrees Celsius, 5% CO2 for two hours. The excess CELLstart was aspirated. Next, we prepared 25 mL of the StemPro hESC SFM by combining 22.7 mL DMEM/F-12+Glutamax, 0.5 mL StemPro hESC SFM Growth Supplement, 1.8 mL Bovine Serum Albumin 25%, 20 uL FGF-basic and 45.5 uL 2-Mercaptoethanol. This hESC serum free medium was thoroughly mixed and warmed to 37 degrees Celsius. We then thawed a vial of approximately 2 million cryopreserved human embryonic stem cells in a 37 degree water bath. The cells were emptied into a 15 mL conical tube and warm hESC medium was added in a dropwise fashion until 10 mL of solution was reached. We spun the cells in a centrifuge for 4 minutes at 200×g, aspirated the supernatant and resuspended the cells in 2 mL hESC medium. 250 uL of this solution was applied to each of the six wells with a CELLstart-coated gold electrode. The hESCs were allowed to attach to the electrodes overnight in a 37 degree Celsius 5% incubator. The gold electrodes were then carefully loaded into a 4 mm cuvette with tweezers. 100 uL of Total Human Liver RNA was thawed in a 37 degree Celsius water bath and applied to the 4 mm cuvette. The cuvette was loaded into a Bio-Rad Gene Pulsar XL and we conducted three electrical pulses of 120V for 1500 ms at 500 uF, and infinite resistance. The hESCs plated on the gold electrodes were delicately removed from the cuvette with tweezers and returned to the individual wells. The plate was incubated at 37 degrees for six hours. In one of the liver trials, the hESC cells began to resemble liver cells. They became individualized rather than clumping together, and took on an elongated and brownish appearance compared to clear, round hESCs.

### Section A

### RNA Isolation, Purification and Amplification Protocol

### Isolation of Total RNA

1) Cells in Suspension: Centrifuge at 500×g for 5 minutes. Aspirate the supernatant.
2) Flick the tube to loosen the pellet. Add 350 ul of TRK Lysis Buffer
3) Homogenize the sample by passing the lysate through a 19-21 gauge needle 5-10 times.
4) Add 350 ul of 70% Ethanol to the lysate and mix thoroughly by vortexing.
5) Apply the sample to an RNA spin column in a 2 mL collection tube (max capacity 800 uL).
6) Centrifuge at 10,000×g for 60 seconds at RT.
7) Remove collection tube, discard flow through, and reattach to column. (If needed, repeat steps 5 & 6 with remaining sample)
8) Pipette 500 uL of RNA Wash Buffer I directly onto the spin column.
9) Centrifuge at 10,000×g for 60 seconds at RT.
10) Remove collection tube, discard flow through, and reattach to column.
11) Add 500 uL of RNA Wash Buffer II (diluted with 20 mL 100% EtOH) to the column.
12) Centrifuge at 10,000×g for 60 seconds at RT.
13) Remove collection tube, discard flow through, and reattach to column.
14) Repeat steps 11-13 (i.e. wash with Buffer II again)
15) Centrifuge the empty 2 mL collection tube and column at max speed for 2 min.
16) Transfer the column into a clean 1.5 mL centrifuge tube.
17) Preheat DEPC-treated water to 70 degrees C.
18) Add 70 uL DEPC-treated water directly onto the spin column.
19) Incubate the column and water for 5 minutes at room temperature.
20) Centrifuge at 10,000×g for 1 minute. The Total RNA is eluted in the column.
   Isolation of miRNA
1) Assemble filtration column into a collection tube (Invitrogen kit)
2) Pipette 70 uL Total RNA in DEPC-treated water onto the center of the column. Do not touch the membrane with the pipette tip.
3) Centrifuge at 13,000×g for 6 minutes. The eluate in the tube is the miRNA. The RNA on the column is the mRNA.
   Amplification of miRNA

Polyadenylation
1) Aliquot 18 ul of eluate into a microcentrifuge tube OR concentrate the 70 uL sample into 18 uL total volume using a SpeedVac Concentrator at low heat, and transfer to a microcentrifuge tube.
2) Dilute the 10 mM ATP1:5000 in 1 mM Tris
3) Add the following at room temperature: 18 uL miRNA+2.5 uL 10×miRNA Reaction Buffer+2.5 uL 25 mM MnCl₂+1 uL Diluted ATP+1 uL Poly A Polymerase
4) Mix and centrifuge briefly
5) Incubate at 37 degrees C. for 15 minutes

Reverse Transcription
1) Dilute Oligo (dT)24V Primer 1:10 in 0.1×TE Buffer. Only 2 uL of diluted Oligo will be needed.
2) Briefly centrifuge the 25 uL miRNA tube and place on ice.
3) Add 2 uL of the diluted Oligo(dT) 24V primer to the tube on ice. Mix and briefly centrifuge.
4) Incubate at 65 degrees C. for 10 minutes. Immediately transfer the tube to ice for 2 minutes.
5) Briefly vortex and centrifuge the following reagents before adding them to the tube on ice: 10 uL 5×First-Strand buffer+5 uL 0.1M DTT+2.5 uL 10 mM dNTP Mix+1 uL RNase OUT+2 ul SuperScript III RT+2.5 uL DEPC-treated water
6) Mix the tube gently (do NOT vortex or centrifuge). Incubate at 46 degrees C. for 1 hour.
7) Add 8.75 uL of 0.5M NaOH/50 mM EDTA (this stops the rxn. A brown color is normal).
8) Briefly vortex and centrifuge.
9) Incubate the tube at 65 degrees C. for 30 minutes (this degrades the miRNA)
10) Add 12.5 uL of 1M Tris (this neutralizes the rxn).
11) Briefly vortex and centrifuge.
12) Add 28.75 uL of 1×TE buffer to bring the total volume to 100 uL

Purification of First-Strand cDNA
1) Assemble a cDNA filtration Column into a cDNA Ultrafiltration Tube
2) Pipette 100 uL of cDNA (from step 12 above) onto the center of the column (do NOT touch the membrane with the pipette tip)
3) Centrifuge at 13,000×g for 6 minutes (align properly)
4) Add 200 uL of 1×TE buffer to the column. Pipette the buffer up and down without touching the membrane ˜5 times.
5) Centrifuge at 13,000×g for 6 minutes.
6) Separate the column from the tube. Discard the flow-through. Insert the column into the same tube.
7) Add 200 uL of 1×TE buffer to the center of the column. Gently pipette the buffer up and down ˜5 times.
8) Centrifuge at 13,000×g for 6 minutes.
9) Separate the column from the tube and discard the tube and flow-through.
10) Add 5 uL of 10 mM Tris to the column and gently tap the side to mix it.
11) Place the column upside-down in a new cDNA ultrafiltration tube.
12) Centrifuge at 13,000×g for 3 minutes. The eluate is purified cDNA.
13) Make sure the volume of cDNA (eluate) is 10 uL. If not, add the remainder with DEPC-treated water to obtain a final volume of 10 uL.

Tailing of First-Strand cDNA
1) Heat the cDNA (from step 13 above) at 80 degrees C. for 10 minutes.
2) Chill on ice for 2 minutes, briefly centrifuge, then return to ice.
3) In a separate RNase-free tube, add the following reagents: 2 uL 10×miRNA Reaction buffer+4 uL 10 mM dTTP+2 uL Terminal Deoxynucleotidyl Transferase+2 uL DEPC-treated water.
4) Mix gently (do NOT vortex or centrifuge)
5) Add the reaction mix above to the cDNA (combine the tubes) for a final volume of 20 uL. Briefly centrifuge.
6) Incubate at 37 degrees C. for 3 minutes.
7) Heat at 80 degrees C. for 10 minutes (this stops the rxn). Briefly centrifuge, then return to room temperature for 2 minutes.

T7 Promotor Synthesis
1) Add 2 uL T7 Template Oligo to the tailed cDNA (from step 7 above)
2) Incubate at 37 degrees C. for 10 minutes (this anneals the strands)
3) Add the following components to the tube: 1 uL 10×miRNA Reaction buffer+1 uL 10 mM dNTP Mix+1 uL Klenow Enzyme
4) Briefly Centrifuge. Incubate at room temperature for 30 minutes.
5) Heat at 65 degrees C. for 10 minutes (this stops the rxn). Place the tube on ice.

In Vitro Transcription (Important to Use an Air Incubator—No Condensation Ok)
1) Place each of the 100 mM NTPs and T7 Enzyme Mix at room temperature to thaw
2) Warm the 10×T7 Reaction buffer at 37 degrees C. for five minutes in an air incubator. Vortex briefly, then keep at room temperature.
3) Incubate the cDNA (from step 5 above) at 37 degrees C. for 10 minutes (this re-anneals the strands)
4) Add the following components to the tube at RT: 1.5 uL each of 100 mM ATP, CTP, GTP & UTP+4 uL 10×T7 Reaction buffer+7 uL T7 Enzyme Mix
5) Briefly Centrifuge
6) Incubate for 4-6 hours at 37 degrees C. in an air incubator.

Purify senseRNA

Amplification of mRNA

Round One 1^{St} Strand cDNA Synthesis
1) Add the following components to a tube: x ul Total RNA sample (10-500 pg)+1 ul T7-Oligo(dT) Primer B+x ul RNase-Free Water
2) Incubate at 65 degrees C. for 5 minutes
3) Chill on ice for 1 minute
4) Centrifuge Briefly
5) Prepare the following cDNA Synthesis Master Mix: 1.5 uL Reverse Transcription PreMix-SS+0.25 uL DTT+0.25 uL SuperScript III Reverse Transcriptase
6) Add the 2 uL of Master Mix to the tube with Total RNA.
7) Incubate at 50 degrees C. for 30 minutes

Round One 2^{nd} Strand cDNA Synthesis
1) Prepare the following 2^{nd} strand cDNA Synthesis Master Mix: 4.5 uL DNA Polymerase PreMix-SS1+0.5 uL DNA Polymerase-SS 1
2) Add 5 uL (the entire sample) of 2^{nd} strand Master Mix to the tube with the Total RNA
3) Incubate at 65 degrees C. for 10 minutes
4) Centrifuge Briefly
5) Incubate at 80 degrees C. for 3 minutes
6) Centrifuge Briefly
7) Chill on Ice
8) Add 1 uL cDNA Finishing Solution-SS
9) Incubate at 37 degrees C. for 10 minutes
10) Transfer directly to incubation at 80 degrees C. for 3 minutes
11) Centrifuge Briefly
12) Chill on Ice

Round One In Vitro Transcription
1) Warm T7 RNA Polymerase, In Vitro Transcription PreMix A, T7 Transcription Buffer, DTT and RNase-Free DNase I to room temperature
2) Thoroughly mix T7 Transcription Buffer (heat to 37 degrees if a precipitate is present)
3) Prepare In Vitro Transcription Master Mix: 4 uL T7 Transcription Buffer+27 uL In Vitro Transcription PreMix A+4 uL DTT+4 uL T7 RNA Polymerase
4) Add 39 uL (the entire sample) of In Vitro Transcription Master Mix to the tube with the Total RNA
5) Incubate at 42 degrees C. for 4 hours exactly
6) Add 2 uL of RNase-Free DNase I
7) Incubate at 37 degrees C. for 15 minutes

Round One RNA Purification (Use Qiagen RNeasy MinElute Cleanup Kit)
1) Prepare 350 uL of RLT/β-ME Solution at a ratio of 1 mL Buffer RLT to 10 uL of β-mercaptoethanol
2) Prepare 650 uL of RPE Solution by diluting 1 volume of Buffer RPE with 4 volumes of 100% EtOH.
3) Add the following components to the tube with mRNA: 47.5 uL RNase-Free Water+0.5 uL Poly(I)+350 ul RLT/β-ME Solution+250 uL 100% EtOH
4) Pipette the sample into an RNeasy MinElute spin column in a 2 mL collection tube
5) Centrifuge at 12,000×g for 15 seconds. Discard flow through
6) Pipette 650 uL RPE Solution onto the center of the column
7) Centrifuge at 12,000×g for 15 seconds. Discard flow through
8) Pipette 650 uL 80% EtOH onto the center of the column
9) Centrifuge at 12,000 for 15 seconds. Discard flow through
10) Pop out the MinElute spin column and place it into a new collection tube
11) Centrifuge at full speed for 5 minutes. Discard flow through
12) Transfer the column again into a new 1.5 mL collection tube.
13) Pipette 14 uL RNase-Free Water directly onto the center of the column
14) Incubate at room temperature for five minutes
15) Centrifuge at full speed for one minute (this elutes the RNA)

Round Two 1^{st} strand cDNA Synthesis
1) Add 2 uL of Random Primers-SS to the tube with the mRNA
2) Transfer the sample into a 0.2-0.6 mL sterile reaction tube
3) Adjust the volume of the sample to 3 uL by vacuum centrifugation without heat. Put the same volume as the sample into a separate test tube. Record the length of time it takes for the water sample to condense to 3 uL. Repeat the length of vacuum centrifugation with the RNA sample.
4) Incubate at 65 degrees C. for 5 minutes
5) Chill on ice for 1 minute
6) Centrifuge Briefly
7) Prepare cDNA Synthesis Master Mix in a separate tube on ice: 1.5 uL Reverse Transcription PreMix-SS+0.25 uL DTT+0.25 uL SuperScript II RT
8) Gently mix the tube of cDNA Synthesis Master Mix, then add 2 uL (the entire sample) to the tube on ice.
9) Incubate at room temperature for 10 minutes.
10) Transfer the tube to a waterbath or thermocycler and incubate at 37 degrees C. for 1 hour
11) Add 0.5 uL RNase H-SS
12) Gently mix the tube
13) Incubate at 37 degrees C. for 20 minutes
14) Incubate at 95 degrees C. for 2 minutes
15) Chill on ice for 1 minute
16) Centrifuge Briefly

Round Two 2^{Nd} Strand cDNA Synthesis
1) Add 1 uL T7-Oligo(dT) Primer C
2) Gently mix the tube
3) Incubate at 70 degrees C. for 5 minutes
4) Incubate at 42 degrees C. for 10 minutes
5) Centrifuge Briefly
6) Prepare cDNA Synthesis Master Mix in a separate tube: 13 uL DNA Polymerase PreMix-SS 2+0.5 uL DNA Polymerase-SS 2
7) Gently mix the Master Mix, then add 13.5 uL (the entire sample) to the tube with the cDNA
8) Incubate at 37 degrees C. for 10 minutes
9) Centrifuge Briefly
10) Incubate at 80 degrees C. for 3 minutes
11) Centrifuge Briefly
12) Chill on ice

In Vitro Transcription of Aminoallyl-aRNA
1) Thaw the Transcription PreMix B, T7 RNA Polymerase, T7 Transcription Buffer, Aminoallyl-UTP, DTT and RNase-free DNase I to RT. If the T7 Transcription Buffer has a precipitate, heat it to 37 degrees C. until it dissolves.
2) Prepare the Transcription Master Mix: 9.6 uL RNase-free water+4 uL T7 Transcription Buffer+16 uL Transcription PreMix B+2.4 uL Aminoallyl-UTP+4 uL DTT+4 uL T7 RNA Polymerase
3) Add 40 uL (the entire amount) to the sample.
4) Incubate at 42 degrees C. for 9 hours (if overnight, hold at 4 degrees C. after 9 hrs)
5) Add 2 uL RNase-free DNase I
6) Incubate at 37 degrees C. for 15 minutes

Aminoallyl-aRNA Purification

This step requires the Qiagen RNeasy MinElute Cleanup Kit
1) Prepare 350 uL RLT/β-ME Solution: ratio of 1 mL Buffer RLT to 10 uL of (3-ME
2) Prepare 1.4 mL RPE Solution: dilute 1 volume of Buffer RPE with 4 volumes 100% Ethanol
3) Add to the sample: 38 uL RNase-free water+350 uL RLT/β-ME Solution+250 uL 100% Ethanol
4) Apply the sample to an RNeasy spin column in a 1.5 mL collection tube
5) Centrifuge at 12,000×g for 15 seconds
6) Discard flow-through
7) Apply 700 uL RPE Solution onto the column
8) Centrifuge at 12,000×g for 15 seconds
9) Discard flow-through. Reattach 1.5 mL tube to the column.
10) Apply another 700 uL RPE Solution onto the column
11) Centrifuge at 12,000×g for 2 minutes
12) Discard flow-through Reattach 1.5 mL tube to the column,
13) Centrifuge dry column at full speed for 1 minute
14) Discard flow-through
15) Transfer the RNeasy spin column to a fresh 1.5 mL collection tube
16) Apply 25 uL (can be more but not less) of RNase-free water directly to the column
17) Incubate at RT for 5 minutes
18) Centrifuge at 12,000×g for 1 minute to collect the aminoallyl-aRNA

### Section B

### Cryopreserved Human Hepatocyte Thawing and Plating Protocol (CellzDirect)

1) Watm CHRM and supplemented Thawing/Plating Medium prior to thawing cryovial of human hepatocytes
2) Transfer cryovial from dewar to laboratory and immediately thaw in the 37 degree Celsius water bath until all ice is liquefied.
3) Immediately remove vial from water bath, spray with 70% alcohol, wipe dry and place in a biosafety cabinet.
4) Quickly pour the contents of the vial into the tube of CHRM. Rinse empty vial with a small amount of CHRM and then pour back into the CHRM tube.
5) Mix gently and centrifuge at 100×g for 10 min at room temperature.
6) Aspirate the supernatant taking care not to disturb the pellet.
7) Add 3 ml of Thawing/Plating Medium to the tube and resuspend cells by gently swirling and rocking the tube.
8) Dispense 500 uL of cell suspension into each of six wells in a 24 well plate and disperse cells evenly across the bottom of the culture wells. Use a figure “8” then a “criss-cross” motion. Do not swirl.
9) Allow cells to attach for approximately 4-6 hours before moving or washing plates.
10) Agitate plates to release any debris and unattached cells and aspirate the medium.
11) Replace with 500 uL of 37 degree Celsius Incubation Medium per well.
12) Return the cells to a 37 degree Celsius incubator and allow recovery overnight.

### SCOPE OF THE INVENTION

The above presents a description of the best mode I contemplate of carrying out my method and apparatus and of the manner and process of making and using them, in such full, clear, concise, and exact terms as to enable any person skilled in the art to which they pertain to make and use my method and apparatus. My method and apparatus is, however, susceptible to modifications and alternate constructions from the illustrative embodiment discussed above which are fully equivalent. Consequently, it is not the intention to limit my method and apparatus to the particular embodiment disclosed. On the contrary, my intention is to cover all modifications and alternate constructions coming within the spirit and scope of my method and apparatus as generally expressed by the following claims, which particularly point out and distinctly claim the subject matter of my invention:

1. A method of converting host cells of a first type into cells of a second type by placing the host cells in intimate contact with donor cells of the second type, said intimate contact being under predetermined conditions that transport a sufficient number of mRNA molecules from the donor cells into the host cells to reprogram the host cells into said second type, said conditions including the application of an electromagnetic force.

2. The method of claim 2 where said predetermined conditions include a media that enables the electromagnetic force to produce an electrophoresis effect that acts on the mRNA molecules from the donor cells to transport them through an outer cell membrane into the hosts cells.

3. The method of claim 1 where said predetermined conditions include subjecting the host and donor cells while in intimate contact to a transporting force that enables the mRNA molecules of the donor cells to penetrate an outer membrane wall of host cells without damaging said membrane wall.

4. The method of claim 3 where transporting force includes an electric field.

5. The method of claim 3 where transporting force includes a magnetic field.

6. The method of claim 1 where transporting force includes a combined electric field and magnetic field.

7. A method of converting host cells of a first type into cells of a second type by placing the host cells in intimate and direct contact with an activated ova in a matrix and subjecting the matrix to electrophoresis to transport an array of reprogramming substances being produced by the activated ova across an outer membrane of individual host cells into the host cell to reprogram the host cells.

8. A method of transferring a biologically active material to host cells, said method comprising the steps of
(a) placing the biologically active material on a first electrode of a pair of electrodes and placing on a second electrode of the pair of electrodes the host cells, and
(b) positioning the electrodes closely together so there is a narrow gap between the electrodes, and
(h) applying an electrical field across said closely spaced electrodes that has sufficient strength so that the biologically active material migrates across the gap and into the host cells on the second electrode.

9. The method of claim 8 where the gap that does not exceed 50 microns.

10. The method of claim 8 where the gap is substantially from 15 microns to 5 millimeters.

11. The method of claim 8 where the strength of the electric field is substantially from 50 to 150 volts.

12. The method of claim 8 where a static direct electric field is applied across the electrodes.

13. The method of claim 8 where a pulsating direct electric field is applied across the electrodes.

14. The method of claim 8 where a magnetic field is applied across said gap concurrent with the application of the electric field, the direction of the magnetic field being substantially at a right angle to the direction of migration across the gap of the biologically active material.

15. The method of claim 8 where a gel is disposed between the electrodes.

16. The method of claim 8 where the biologically active material is applied as a thin coating carried by the first electrode that has a thickness substantially from 0.015 to 4.0 millimeters and the host cells are applied as a thin coating carried by the second electrode that has a thickness substantially from 0.015 to 4.0 millimeters, said coatings being substantially planar and facing each other and said first electrode having a negative polarity and said second electrode having a positive polarity.

17. The method of claim 8 where the biologically active material comprises activated ova that produces a mixture mRNA and miRNA in predetermined proportions.

18. The method of claim 8 where the biologically active material comprises a mixture of mRNA and miRNA molecules that are amplified in number from those originating from the activated ova, said amplified in number of mRNA and miRNA molecules being substantially in the same proportions as normally yielded by the activated ova.

19. A method of changing differentiate host cells into pluripotent cells comprising the steps of
(a) extracting mRNA and miRNA from an activated ova of a living organism when the ova is reprogramming its nucleus,
(b) amplifying the number of molecules of mRNA and miRNA extracted from step (a),
(c) processing the mRNA and miRNA from step (b) by polyadenylation,
(d) placing the polyadenylated mixture of mRNA and miRNA on a negative electrode of a pair of electrodes and placing on a positive electrode of the pair of electrodes differentiate host cells to be transformed into the pluripotent cells, and
(e) positioning the electrodes closely together so there is a narrow gap between the electrodes, and
(f) applying an electrical field across said closely spaced electrodes that has sufficient strength so that the mRNA and miRNA migrate across the gap into the host cells on the second electrode to interact therewith to transform the host cells into the pluripotent cells.

20. The method of claim 19 where, subsequent to step (c) and prior to step (d), the mRNA and miRNA molecules are blended in predetermined proportions substantially in the same proportions as normally yielded by the activated ova.

21. The method of claim 19 where the ova is chemically, electrically or mechanically stimulated to make the mRNA and miRNA.

22. The method of claim 19 where the mRNA and miRNA is extracted using a centrifuge.

23. The method of claim 19 where the mRNA and miRNA are charged negatively so they migrate to the positive electrode.

24. The method of claim 19 where in step (b) the mRNA and miRNA are hydrolyzed in an aqueous solution, purified and freeze-dried prior to step (c).

25. The method of claim 19 where a gel material is within the gap, said gel material being selected from the group consisting of agarose, Matrigel, and acrilamide.

26. The method of claim 19 where the gel material includes an electrolyte.

27. The method of claim 19 conducted without the use of harmful substances that would impede clinical use.

28. The method of claim 19 where the gap that does not exceed 50 microns.

29. The method of claim 28 where the gap is substantially from 15 microns to 5 millimeters.

30. The method of claim 19 where the strength of the electric field is substantially from 50 to 150 volts.

31. The method of claim 19 where a static direct electric field is applied across the electrodes.

32. The method of claim 19 where a pulsating direct electric field is applied across the electrodes.

33. The method of claim 19 where a magnetic field is applied across said gap concurrent with the application of the electric field, the direction of the magnetic field being substantially at a right angle to the direction of migration across the gap of the biologically active material.

34. The method of claim 19 where a gel is disposed between the electrodes.

35. The method of claim 19 where the mRNA and miRNA mixture is applied as a thin coating on the negative electrode and the host cells are applied as a thin coating on the positive electrode, said coatings facing each other and in intimate contact when the electric field is applied.

36. A method of making pluripotent cells comprising the steps of
(a) placing an activated ova on a first electrode of a pair of electrodes and placing on a second electrode of the pair of electrodes differentiated host cells to be converted into the pluripotent cells, said activated ova producing mRNA and miRNA in substantially the same proportions as it does when it is activated within the body of a donor of the ova,
(b) positioning the electrodes closely together so there is a narrow gap between the electrodes, and
(c) applying an electrical field across said closely spaced electrodes that has sufficient strength so that the mRNA and miRNA being produced by the activated ova migrate across the gap and into the host cells on the second electrode.

37. A method of separating the constituents of a mix of reprogramming substances including reprogramming proteins, mRNA and miRNA comprising the steps of
(a) providing a gel within a light-transmitting container that has a closed end and an open end.
(b) including makers within the container that identify by color separate zones along the length of the container corresponding to collection of separated constituents, and
(c) under cryogenic temperature conditions, placing the mix in contact with the gel near the open end and centrifuging the container so the constituents separate and collect in different layers within the gel according to their mobility.

38. An apparatus for transferring biologically active material to host cells comprising
a support mounting member,
a pair electrodes attached to the mounting member, said electrodes having opposed planar surfaces facing each other, at least one electrode moveably mounted so the electrodes have (a) a first, spaced apart position to enable the biologically active material to be placed on the planar surface of one electrode and the host cells to be placed on the planar surface of the other electrode, and (b) a second position where the electrodes are close together so there is a gap between the electrodes that does not exceed 50 microns, and
a power source that applies an electrical field across said electrodes when in the second position that has sufficient strength so that the biologically active material migrates across the gap into the host cells on the second electrode.

39. The apparatus of claim 38 where the electrodes are substantially parallel plates.

40. The apparatus of claim 38 where a magnetic field is applied across said gap concurrent with the application of the electric field, the direction of the magnetic field being substantially at a right angle to the direction of migration across the gap of the biologically active material.
